(19) United States
(12) Patent Application Publication
(10) Pub. No.: US 2024/0082021 A1
(43) Pub. Date: Mar. 14, 2024

(54) DR SHRINAND V VAIDYA'S PEGLESS FEMORAL TEMPLATE TRIAL

(71) Applicants: Shrinand VAIDYA, Mumbai (IN); Chintan VAIDYA, Mumbai (IN)

(72) Inventors: Shrinand VAIDYA, Mumbai (IN); Chintan VAIDYA, Mumbai (IN)

(21) Appl. No.: 17/767,896

(22) PCT Filed: Mar. 6, 2020
(86) PCT No.: PCT/IN2020/000009

§ 371 Date: Apr. 10, 2022

(30) Foreign Application Priority Data
Oct. 10, 2019 (IN) 201921040898

(51) Int. Cl. A61F 2/46; A61F 2/38

(52) U.S. Cl. CPC A61F 2/4684; A61F 2/3859; A61F 2002/3895; A61F 2310/00017

(57) ABSTRACT

Achieving proper mechanical axis and rotational position of femoral component perpendicular to the tibial component throughout range of motion is necessary for long term successful outcomes of unicondylar knee replacement. Dr. Shrinand V Vaidya's pegless template trial-P.T.T. is an orthopaedic surgical device used to determine mechanical axis, rotation and size of the femoral component. P.T.T. has two holes in anterior part which matches with femoral finishing block of respective size. P.T.T. has central watermark to determine final position of the femoral component. Position of P.T.T. is guided by tibial trial through range of motion and final position is marked over femoral condyle just anterior to the central watermark over P.T.T. Alternatively pins are inserted through the holes in PTT and removed making two holes which can be used for alignment of femoral finishing block.

### FIELD OF INVENTION

The present invention deals with Orthopaedic surgical device useful in knee replacement surgery and particularly deals with a device (Pegless femoral Template Trial—P.T.T.″) for implanting femoral (thigh bone) component in unicondylar (only half) knee replacement surgery.

### BACKGROUND OF THE INVENTION

Knee Replacement Surgery:

Arthroplasty is an Orthopaedic surgery where articular surface of joint is replaced, remodelled or realigned. It is a procedure to relieve pain and restore the function of the joint after damage, usually secondary to arthritis.

The procedure involves cutting away damaged bone and cartilage from thighbone (femur), shinbone (Tibia) and kneecap (patella) and replacing it with an artificial joint (prosthesis) made of metal alloys, high-grade plastics and polymers.

Knee replacement surgery can help patients whose knee has degenerated due to osteoarthritis, rheumatoid arthritis, or post-traumatic arthritis (when an injury has damaged the knee)

The knee is divided into three major compartments:
Medial compartment (the inside part of the knee)
Lateral compartment (the outside part)
Patellofemoral compartment (the front of the knee between the kneecap and thighbone)

Osteoarthritis can affect any of, the three compartments. Total knee arthroplasty is considered as gold standard for symptomatic arthritis of knee joint.

Total Knee Arthroplasty:

Distal surface of femur (thigh bone) and proximal surface of tibia (shin one) is replaced by metal or plastic components in total knee arthroplasty. The surgeon caps the ends of the bones that form the knee joint with metal or plastic components, or implants a prosthetic, shaped as a joint. This enables the knee to move properly.

Total knee replacement provides freedom from pain, improved mobility, improved quality of life because everyday activities and exercise are easier.

Evolution of knee arthroplasty, which has a history of almost 50 years, involves repetitious cycles of failure and development. During its early stage (1970-1974), instruments of the unicondylar, duocondylar, or hinged types were used, but these were eventually abandoned due to low success rates.[1] A replacement for the total condylar type was successfully developed and became the model for total knee arthroplasty.[1] Insall et al. added a cam to the femoral prosthesis and a post to the tibial prosthesis for posterior cruciate ligament substitution knee arthroplasty to accelerate the

posterior location of the femoral prosthesis when flexed at about 70 degrees, thus enhancing flexion.[2] These Insall-Burnstein and kinematic interpositions became the foundation of modern

knee arthroplasty.[1]

Partial Knee Replacement(Unicondylar Knee Arthroplasty):

A partial knee replacement is an alternative to total knee replacement for some patients with osteoarthritis of the knee involving single compartment. ‘Unicompartmental knee replacement(UKR)’ replaces only one compartment which is diseased at a time and preserves the native ligaments and soft tissue which stabilize knee joint.

Comparisons of Total Knee Replacement and Partial Knee Replacements: Pain, Complications, and Risk—in Knee Relacement Surgeries:

Total knee arthroplasty has more morbidity in term of restricted range of motion, more bone resection, more blood loss and longer time of rehabilitation as compared unicondylar knee replacement is done through a smaller incision, less bone resection, preserves the cruciate ligaments, patients usually spend less time in the hospital and return to normal activities sooner than total knee replacement patients.[3]

Acceptance and Challenges in Partial Knee Replacement(Unicondylar Knee Arthroplasty):

Results of unicompartmental knee arthroplasty remained controversial when it was introduced. Several authors reported unsatisfactory results for unicompartmental knee arthroplasties but over the next decade, better instrumentation, better surgical techniques and proper patient selection improved successful outcomes.[4] Along with minimally invasive techniques, unicompartmental knee arthroplasty has aroused much interest.[5]‘Unicondylar knee arthroplasty (UKA)’ has gained increased popularity as a less-invasive alternative to total joint arthroplasty (TKA) for the treatment of localized symptomatic osteoarthritis.

The success of this procedure is measured by pain relief, improved function, patient satisfaction and implant longevity. Implant longevity is dependent on prosthetic factors such as implant size, tribology(The study of friction, wear, lubrication, and the design of interacting surfaces in relative motion), geometry, alignment, and position and surgical factors including surgical skill and experience, duration of surgery, appropriate preparation, and implantation of the prosthesis. [6]

Patient factors including size, weight, activity, the existence of medical comorbidities, psychological, and physiological response to joint surgery also plays role.

The correct unicondylar knee arthroplasty implant system must maximize the chance of procedural success and must minimize the chance of failure by preventing malposition of components and malalignment in varus (the bone segment distal to a joint angled inward, that is, angled medially, toward the body's midline) or valgus (the bone segment distal to a joint angled outward, that is, angled laterally, away from the body's midline.)[7]

There is a constant direction of stress applied to the tibial plateau(along the mechanical axis of tibia) but the stress applied to the distal femur is varied. The force is applied at the joint surface and its direction is always perpendicular both to the joint surface and to the horizontal plane, even when the tibial component is placed in varus or valgus alignment.

The components should be shaped to allow distracting, sliding and rolling movements between the bones. The components should apply only compressive stress to the juxta-articular bone. All surviving soft tissues should be kept and restored to their natural tension.[8] The areas of contact between the two components of the prosthesis should be large enough and congruent to maintain the pressure under load at a level which the prosthetic materials can withstand. [8] The prosthesis should apply only evenly distributed compressive forces to the tibial bone.

If the components are to bear their loads through large areas of contact, they must fit one another in all postures of the joint—and the only shapes which will do so are spheres in spherical sockets i.e. ball and socket joint. However, if two of these were used either side of the joint then only one axis of motion would be possible as the mechanics of the ligaments would not allow movement in another direction.[8] Use of a closely fitting unconstrained spacer trapped by its shape between the rounded femoral component and the flat tibial component enables the maximum contact surface area while enabling a full range of movement. [8]

Tibial component coverage of cut bone surface is important in long term successful outcomes. Poor tibial coverage, i.e. underhang, has been attributed to increased risk of tibial component loosening and subsidence as cancellous bone(soft inner part of bone) cannot bear body weight transmitted through tibial component. Tibial overhang significantly increases risk for residual pain. In addition, overhang can result in putting increased stress on the MCL(medial collateral ligament)[9] It has been proposed that preservation of the joint line and the sagittal J-curve of femur provide opportunity to preserve normal joint function, with potential to result in more normal knee kinematics.[10]

Malpositioned femoral components can cause loosening and early failure because of shearing forces. Finite element analysis of the contact stresses shows that the best position for the femoral component is the centre of the distal femoral condyle in UKA.[11] The femoral component position could be one of the sensitive factors that influence the contact stresses on the PE insert and articular cartilage in UKA.[11]

Giles Scuderi suggested there should be at least 1-2 mm gap around femoral component to prevent overhanging and resulting soft tissue impingement. Femoral component should be 1-2 mm away from sulcus terminalis in front to ensure optimal coverage and staying outside of patellar track[12] If component is positioned anterior patients have patellar impingement and increased pain while on stairs and rising from chairs.[13]

Importance of Restoring Accurate Mechanical Axis Alignment:

Medial femoral condyle is angulated in axial plane so this might be cause incorrect rotatory positioning of the femoral component. (FIG. 2) Femoral component rotatory position should be perpendicular to the tibial component. [13] This implies that the femoral component should not be positioned anatomically on the condyle if it is to be perpendicular to the tibial component.

Achieving proper mechanical axis alignment is important for long term implant survival and disease progression. Studies have reported that ‘slightly under-corrected’ i.e. mild valgus alignment UKAs result in less long-term progression of disease and poly wear.[14]

Earlier ‘Free hand technique’ was used to resect bone cuts which was not accurate and resulted in malposition of components.

Modified guide instruments derived from Lotus prosthesis came into use to obtain better position of tibial prosthesis in middle of 1977.[15]

Local template for femoral and tibial component came into use in association with guide instruments for alignment of the components respect to each other but it did not determine position of components in relation to the mechanical axis of femur and tibia.

Overtime designs improved and accurate bone cutting jigs were developed based on long axis of femur and tibia.

Oxford Knee has spoon for measuring femoral size. This spoon attaches tibial jig for tibial cut. After that milling is done with respective fitting size.

Current Practice:

Current practice to determine the rotation of femoral component in unicondylar knee arthroplasty is to mark vertical line with pen/electrocautery over distal femur directly over midpoint of tibial trial and to determine anterior extent of femoral trial transverse line is made along anterior border of tibial trial. (FIG. 6) This is crude, as marks with cautery or pen may not be visible in case of bleeding form cancellous bone.

Femoral finishing block is aligned according to these marks. It is essential that the sulcus between the apexes of the block is aligned with the vertical line, and it should not extend superior to the horizontal line. (FIG. 7) This method of determining position of femoral component is not efficient.[19]

Cartier et al. suggested that Centering of femoral component on the tibial component throughout the range of motion especially near extension is one of the main feature in unicompartmental knee arthroplasty.^{[21]}

Roberto Rossi et al. suggested ‘The Range of motion technique’ in which the knee is manipulated through a full arc of motion several times, allowing the tibial tray to float and orientate itself in the best position relative to the femoral component.

With the knee in full extension, the final position of the selfaligned tibial component is visualized and marked on the anterior cortex of the tibia.^{[22]}

As cited in above paragraphs though dramatic advancements have been made in the field of knee replacement, alignment and rotation of femoral component still remains an elusive issue which may have a direct bearing on the completes success of knee replacement surgeries.

Though numerous attempts have been made this direction, an optimum, simple consistent and cost effective solution for the same is still to be found.

### OBJECTS AND SUMMARY OF OUR INVENTION

We tried to address the difficulties in mechanical alignment and rotation of femoral component in unicondylar knee replacement surgery. We are offering modification in current practice for improving existing technology. We created Dr. Shrinand Vaidya's Pegless Template Trial (P.T.T.) to solve the technical problem.

The main object of the present invention is to provide mechanical axis of femoral component perpendicular to the tibial component throughout the range of motion. Another object of the present invention is to ensure 1-2 mm gap around femoral component to prevent overhanging.

We created Pegless template trial (P.T.T.) to know final mechanical axis, rotational alignment and size of femoral component before making cuts of femur. P.T.T. is used after tibial cut is taken and tibial component guides position of the P.T.T. throughout range of motion perpendicular to the tibial component.

### BRIEF DESCRIPTION OF THE FIGURES

FIG. 1. Proper size of femoral component having 1-2 mm gap around it to prevent overhanging

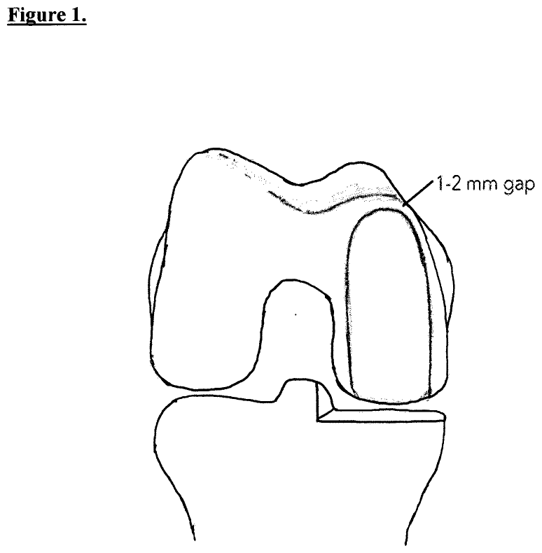

FIG. 2(A) The oval is the anatomic position for the femoral component, and (B) the oval represents the position that is perpendicular to the tibial plateau component. AP, anteroposterior; epi, epicondylar axis; PC, posterior condylar axis; x, posterior condylar resection line.

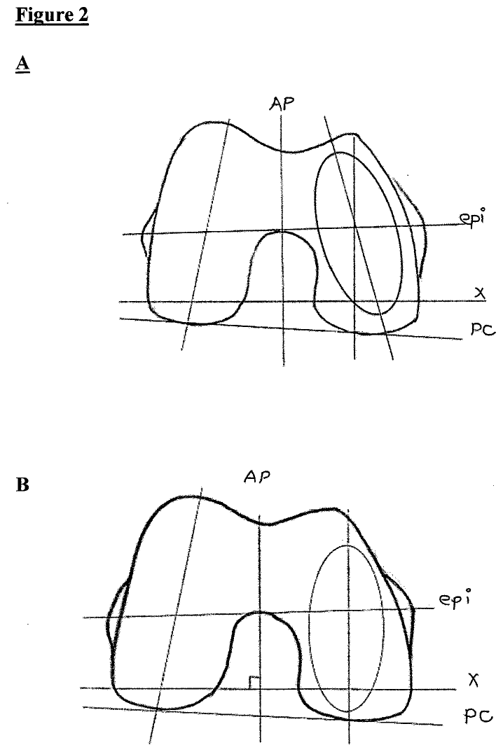

FIG. 3. Extramedullary Tibial jig

FIG. 4. Distal femur cutting block

FIG. 5. Femoral finishing block (1. posterior 105° cut 2. Anterior cut 3. Peg holes 4. Posterior chamfer cut)

FIG. 6. Vertical marking over midpoint of tibial trial to determine rotation and transverse mark to determine extent of the femoral component

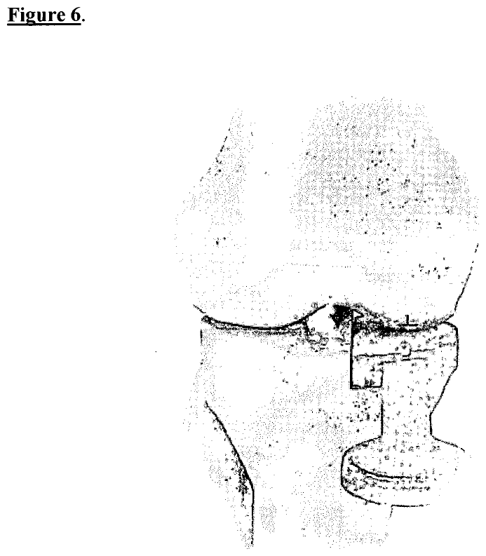

FIG. 7. Sulcus between the apexes of the femoral finishing block is aligned with the vertical line

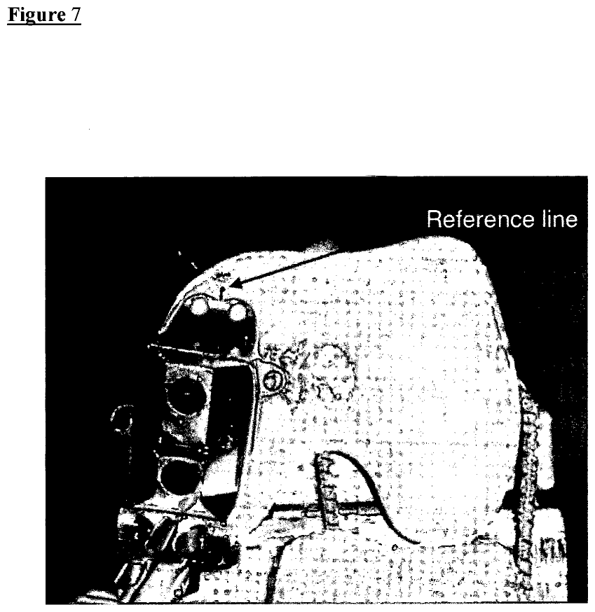

FIG. 8—Front view of P.T.T. showing two 2.5 mm holes in the anterior part of P.T.T. that are 8 mm apart and 4.5 mm from anterior end of P.T.T. and central watermark over P.T.T. Radius of curvature of anterior part is 9 mm

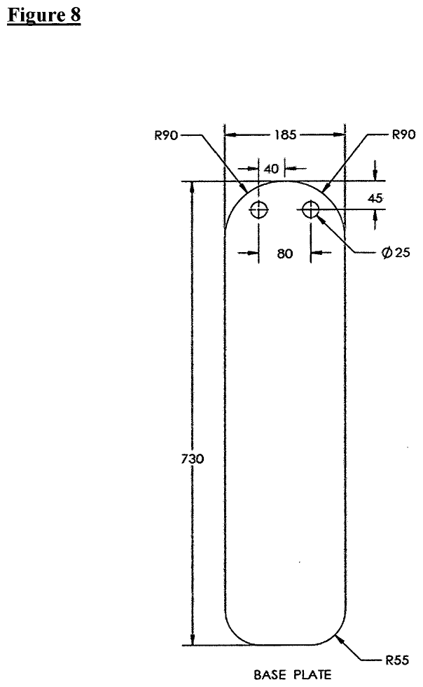

FIG. 9—Side view of P.T.T. showing 1.5 mm thickness and total length of 73 mm

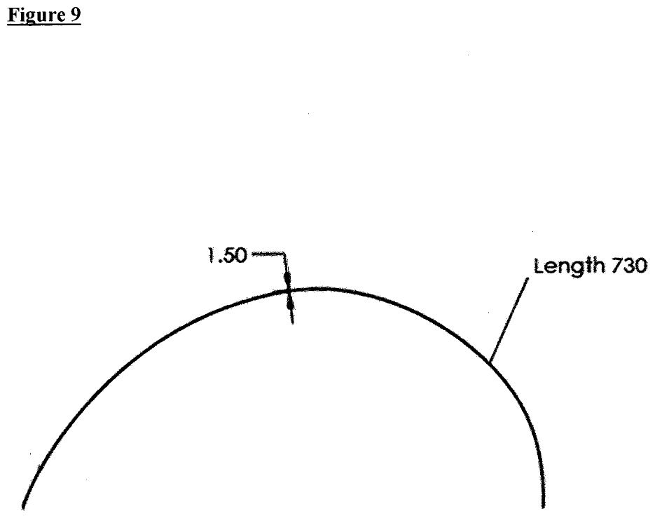

FIG. 10—Top view of P.T.T. showing two 2.5 mm holes.

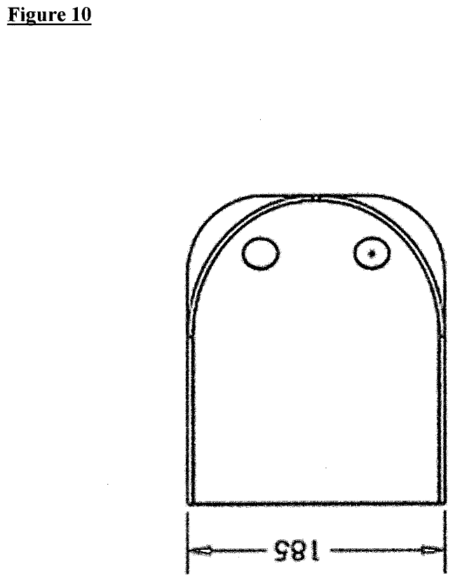

FIG. 11—Bottom view of P.T.T. showing curved posterior end with 5.5 mm radius of curvature on both side.

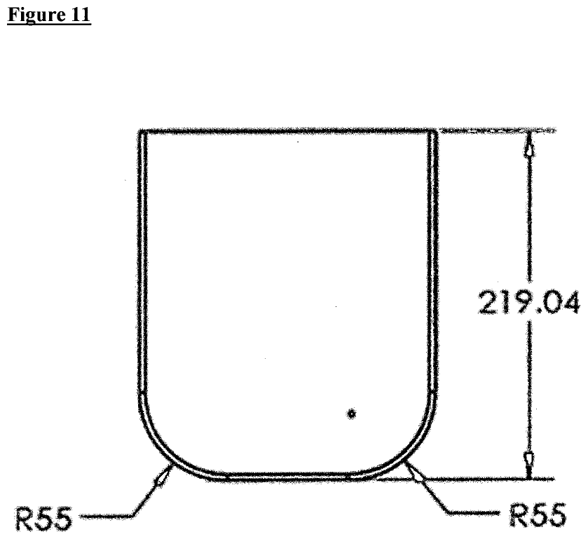

FIG. 12—Front view, side view(left), top view and trimetric view of P.T.T.

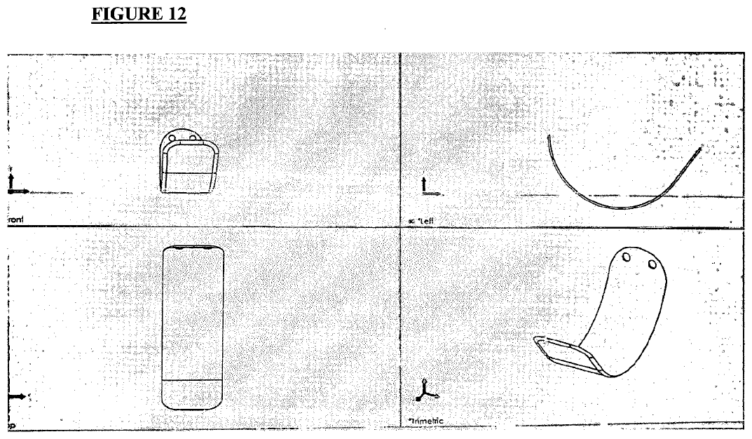

FIG. 13—Size 2 P.T.T. type 2.1.1 showing front, top, bottom and side view with dimensions (mm)

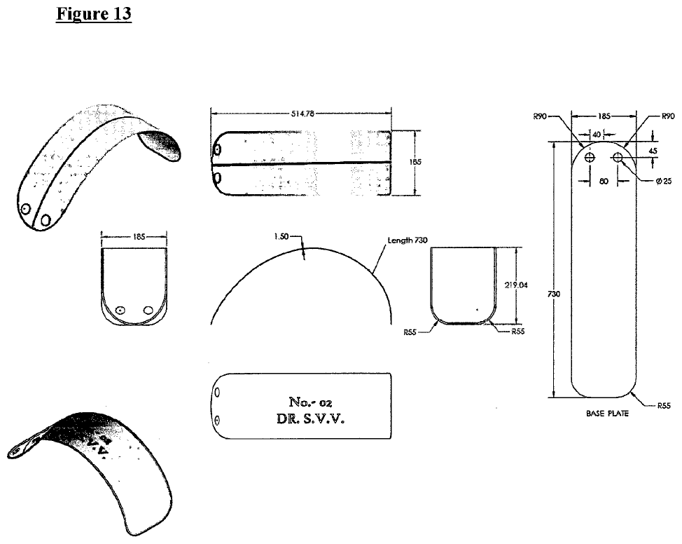

FIG. 14—Size 2 P.T.T. type 2.1.2 showing front, top, bottom and side view with dimensions (mm)

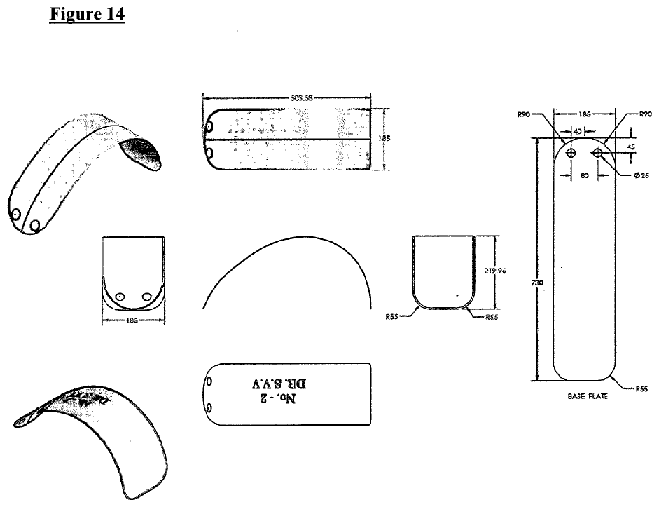

FIG. 15—Size 2 P.T.T. type 2.1.3 showing front, top, bottom and side view with dimensions (mm)

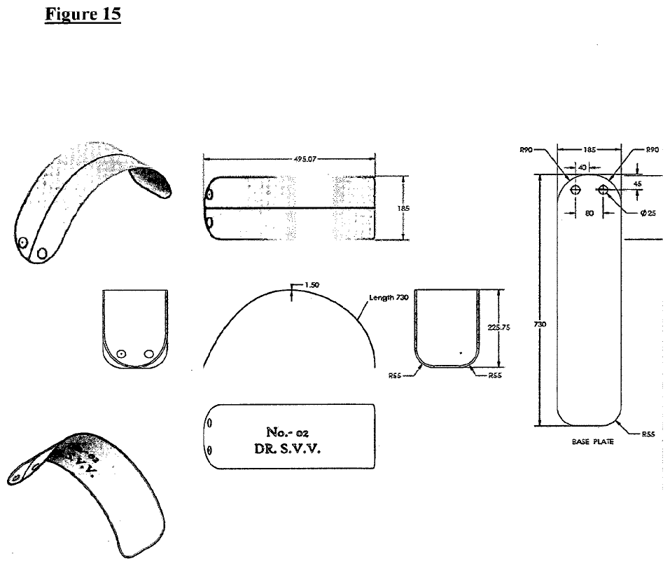

### DETAILED DESCRIPTION OF THE INVENTION

We are presently offering modification specially in relation to the existing Sigma High performance partial knee system (DePuy Synthes—A Johnson & Johnson Company, Warsaw, IN, USA, 2007), widely used world over.

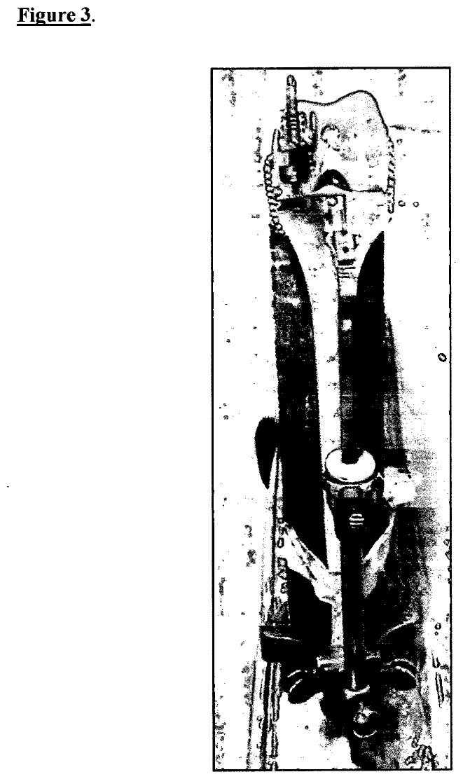

Extra medullary jig (FIG. 3) is used in unicompartmental knee for tibial proximal cut which determines the position of cut perpendicular to tibial mechanical axis by applying clamps over ankle. Jig accommodates tibial cutting block in proximal part and its height can be adjusted to determine thickness of tibial cut.[16] The varus/valgus orientation of the tibial cut can be adjusted by shifting the lower assembly of the ankle clamp from side to side. The lower assembly moves by pressing the varus/valgus wings. The tibial jig uprod and ankle clamp are designed to prevent an adverse reverse slope. The angle of the tibial slope can be adjusted to the patient's natural slope by unlocking the slide locking position and then translating the tibial slope adjuster anteriorly until the desired angle is reached.[17] Leo Whiteside suggested sagittal cut for tibial component to be kept in 10° internal rotation directed toward Anterior superior iliac spine ASIS to accommodate screw home mechanism.

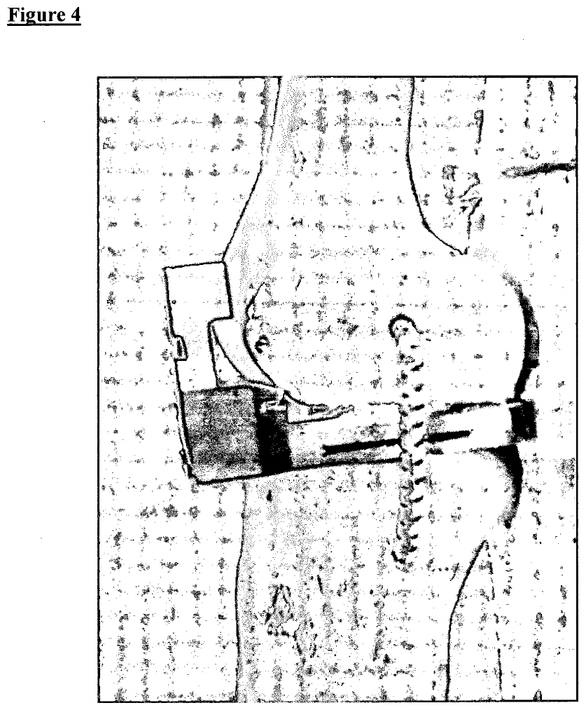

Distal cutting block(FIG. 4) is used to resect distal femoral condyle. Distal femoral resection is done parallel to the tibial resection.[18] It has femoral defect shims and tibial shims for gap balancing which allows to change the thickness of distal femoral cut. Femoral defect shims (1 mm, 2 mm or 3 mm) are used if excessive extension laxity exists relative to flexion. Use of these femoral defect shims will effectively under-resect the distal femur (removing less bone than is replaced by component thickness), tightening the extension gap. If the 7 mm tibial trial is used in balancing, do not add a tibial shim to the tibial side of the distal cutting block. If a thicker tibial trial is used, add the appropriate tibial shim to the tibial side of the distal cutting block.

Alignment guide and extramedullary alignment rod into the slot of the distal femoral cutting block is used to check local alignment, both varus/valgus and flexion/extension. Alignment rod is kept parallel to the intramedullary axis of the femur to achieve proper femoral component position.[18]

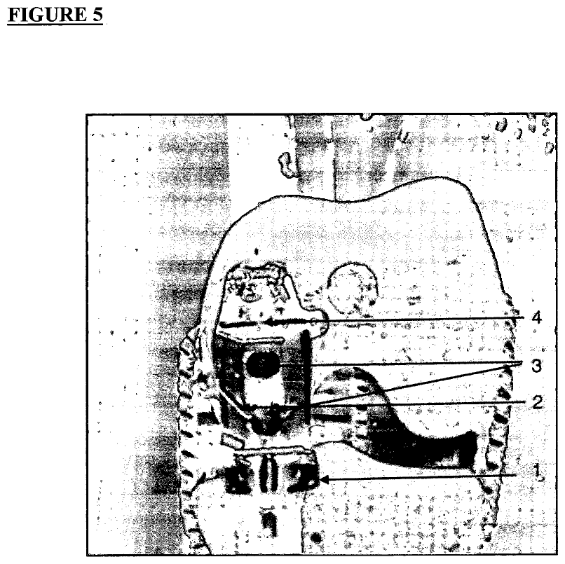

Femoral finishing block(FIG. 5) has three slots in different direction which is used to cut posterior 105° femoral condylar cut, anterior cut and posterior chamfer cut.[19] The leg placed in 90° of flexion. The block size selected during templating is placed under the posterior condyle to be flush with the resected distal femoral surface. Exchange to smaller or larger size is done to find the best fit. The block may be rotated to ensure that the femoral component articulates over the centre point of the tibial component throughout the range of motion. This will increase the likelihood that the tibial component will properly track with the femur in extension and prevent patellofemoral impingement. The femoral finishing block has two holes through which femoral peg drilling is done. Direction of peg holes is not parallel to the mechanical axis of femur this ensures stability of femoral component in arc of motion of the knee.

We applied The Range of motion technique described by Roberto rossi[22] to determine rotation of femoral component based on tibial component but in view of the fact that tibial component cut is taken first in unicondylar knee, it will guide femoral component rotation when repeated extension to flexion movement are performed with trials in unicondylar knee arthroplasty.

Our Invention: Dr. Shrinand V. Vaidya's Femoral Pegless Template Trial—P.T.T.′:

We created ‘Dr. Shrinand V. Vaidya's femoral pegless template trial—P.T.T.’ which is 1.5 mm in thickness. The trial has same sagittal curve and mediolateral dimension as final femoral component of respective size i.e. For size 1 femoral component base plate width—184 mm, height—730 mm, thickness—1.5 mm and Bottom chamfer radius—55 mm. This ensures that femoral template sits over uncut femoral condyle. Dimensions of femoral trial are mentioned in table. Remaining size dimension P.T.T. can also be appropriately devised as and when needed.

| Sigma partial knee | Mediolateral(mm) | Anteroposterior(mm) |
|---|---|---|
| Size 1 | 18.4 | 42.6 |
| Size 2 | 19.4 | 45.1 |
| Size 3 | 20.5 | 47.5 |
| Size 4 | 22.2 | 50.4 |
| Size 5 | 23.7 | 54 |
| Size 6 | 25.2 | 57.5 |

P.T.T. has central watermark which is used as guide to position femoral component perpendicular to the tibial component and in center of the medial femoral condyle. P.T.T. has two 2.5 mm holes matching the position of holes in femoral finishing block and central watermark on P.T.T. that matches with the anterior notch of femoral finishing block. After tibial cut and tibial trial insertion P.T.T. of appropriate size is placed over the femoral condyle.

Optimum final position of P.T.T. throughout range of motion is guided by tibial insert. Rotation is confirmed by ensuring that the watermark is perpendicular to the tibial trial and there is 2 mm gap all around the P.T.T. to ensure that there is no overhanging. It is ensured that femoral component does not fit too anteriorly to prevent patellar impingement. P.T.T. is perpendicular to the tibial insert so that final the tibial component will properly track with final the femoral component. The final position of P.T.T. is marked just anterior to the central watermark over femoral condyle. Two pins are inserted in holes in femoral template trial. This will create two holes on distal femoral medial condyle. These hole will guide the placement of final implant. This method is optional. Slight manual adjustment can be done to ensure proper position of femoral component.

Pins are removed an distal femoral cut is taken with distal femoral cutting block in extension.

It is ensured that sulcus between apexes of the femoral finishing block is aligned with mark over femoral condyle. After that posterior 105° condylar cut of femur is taken followed by posterior chamfer cut and anterior cut and notch cut. Anterior and posterior peg holes are drilled through femoral finishing block.

Femoral finishing block and pins are removed and femoral trial of respective size is inserted in its final position. It is ensured that there is 2 mm gap around final trial and femoral component is perpendicular to tibial component to avoid edge loading and impingement of metal edge and to prevent patellofemoral pain because of anterior overhang. After that tibial trial and final implantation with cementing is done.

While the invention has been described in terms of particular variations and illustrative figures, those of ordinary skill in the art will recognize that the invention is not limited to the dimensions mentioned in the description. Those of ordinary skill in the art will recognize that the dimensions of the PTT can be modified depending on the knee surgery systems offered by different companies and such modifications are in accordance with the variations of the invention. Therefore, to the extent there are variations of the invention, which are within the spirit of the disclosure or equivalent to the inventions found in the claims, it is the intent that this patent will cover those variations as well.

### REFERENCES

1. Song E K, Seon J K, Moon J Y, Ji-Hyoun Y. The evolution of modern total knee prostheses. InArthroplasty-Update 2013 Feb. 20. IntechOpen.
2. Insall J N, Lachiewicz P F, Burstein A H. The posterior stabilized condylar prosthesis: a modification of the total condylar design. Two to four-year clinical experience. J B J S. 1982 Dec. 1; 64(9):1317-23.
3. Jung, Y. B, & Lee, Y. S. Principles of unicompartmental knee arthroplasty. J Korean Orthop Assoc (2004)., 39, 108-114.
4. Laskin, R. S. Unicompartmental tibiofemoral resurfacing arthroplasty. J Bone Joint Surg Am (1978)., 60, 182-185.
5. Repicci, J. A, & Eberle, R. W. Minimally invasive surgical technique for unicondylar knee arthroplasty. J South Orthop Assoc (1999)., 8, 20-27.
6. Jones L, Tsao A, Topoleski L, Chapter 12: Factors contributing to orthopaedic implant wear, Pages 310-350, Wear of orthopaedic implant and Artificial joints.
7. Fehring T K, Odum S, Griffin W L, Mason J B, Nadaud M. Early failures in total knee arthroplasty. Clinical Orthopaedics and Related Research®. 2001 Nov. 1; 392:315-8.
8. Goodfellow J, O'Connor J. The mechanics of the knee and prosthesis design. The Journal of bone and joint surgery. British volume. 1978 August; 60(3):358-69.
9. Chau R, Gulati A, Pandit H, Beard D J, Price A J, Dodd C A, Gill H S, Murray D W. Tibial component overhang following unicompartmental knee replacement—does it matter?. The knee. 2009 Oct. 1; 16(5):310-3.
10. Fitz W. Unicompartmental knee arthroplasty with use of novel patient-specific resurfacing implants and personalized jigs. J B J S. 2009 Feb. 1; 91(Supplement_1):69-76.
11. Kang K T, Son J, Koh Y G, Kwon O R, Kwon S K, Lee Y J, Park K K. Effect of femoral component position on biomechanical outcomes of unicompartmental knee arthroplasty. The Knee. 2018 Jun. 1; 25(3):491-8.
12. Scuderi G, Instrumentation for Unicondylar Knee Replacement, chapter 5: MIS of the hip and the knee A clinical perspective.
13. Hernigou P, Deschamps G. Patellar impingement following unicompartmental arthroplasty. J B J S. 2002 Jul. 1; 84(7):1132-7.
14. Hernigou P, Deschamps G. Alignment influences wear in the knee after medial unicompartmental arthroplasty. Clinical Orthopaedics and Related Research®. 2004 Jun. 1; 423:161-5.
15. Lindstrand A, Boegard T, Egund N, Thorngren K G. Use of a guide instrument for compartmental knee arthroplasty. Acta Orthopaedica Scandinavica. 1982 Jan. 1; 53(4):633-9.
16. Sigma High performance partial knee: Unicondylar surgical technique, page no 4, 9075-21-000 version 1 Revised: 03/12,©DePuy International Ltd. and DePuy Orthopaedics, Inc. 2012
17. Sigma High performance partial knee: Unicondylar surgical technique, page no 5, 9075-21-000 version 1 Revised: 03/12,©DePuy International Ltd. and DePuy Orthopaedics, Inc. 2012
18. Sigma High performance partial knee: Unicondylar surgical technique, page no 12-13, 9075-21-000 version 1 Revised: 03/12,©DePuy International Ltd. and DePuy Orthopaedics, Inc. 2012
19. Sigma High performance partial knee: Unicondylar surgical technique, page no 14-16, 9075-21-000 version 1 Revised: 03/12,©DePuy International Ltd. and DePuy Orthopaedics, Inc. 2012
20. Sigma High performance partial knee: Unicondylar surgical technique, page no 11, 9075-21-000 version 1 Revised: 03/12,©DePuy International Ltd. and DePuy Orthopaedics, Inc. 2012
21. Cartier P, Sanouiller J L, Grelsamer R P. Unicompartmental knee arthroplasty surgery: 10-year minimum follow-up period. The Journal of arthroplasty. 1996 Oct. 1; 11(7):782-8.
22. Rossi R, Bruzzone M, Bonasia D E, Marmotti A, Castoldi F. Evaluation of tibial rotational alignment in total knee arthroplasty: a cadaver study. Knee Surgery, Sports Traumatology, Arthroscopy. 2010 Jul. 1;18(7):889-93

1. An orthopaedic surgical device-‘pegless template trial (PTT)’ to determine mechanical axis, rotational position, medio-lateral centering and size of the femoral component during partial knee replacement surgery and the said device comprising of: a thin metallic plate with a curved anterior end having two holes approximately of 2.5 mm diameter and a curved posterior end and a central watermark running longitudinally over the device and the said device having the same sagittal curve and mediolateral dimension as final femoral component of respective size ensuring that the femoral template sits over uncut femoral condyle and the holes of the said device matching the position of holes in femoral finishing block and the central watermark matching with the anterior notch of femoral finishing block enabling positioning of femoral component perpendicular to the tibial component and in centre of the medial femoral condyle.

2. The orthopaedic surgical device of claim 1, wherein the dimensions of the device are as follow for Size 1 component: total length—73 mm, thickness—1.5 mm, radius of the curved anterior end 9 mm, radius of the curvature of the curved posterior end 5.5 mm on both sides, distance between the holes 8 mm, distance of the holes from the anterior end, 4.5 mm.

3. The orthopaedic surgical device of claim 1, wherein the metal used in making of the device is stainless steel.

4. A method of using the orthopaedic surgical device—‘pegless template trial (PTT)’ of claim 1, comprising the steps of:
a. putting the pegless template trial (PTT) of appropriate size based on the surgical implant system being used over the femoral condyle after tibial cut is taken and tibial trial insert is positioned;
b. moving the knee through repetitive extension to flexion multiple times to see it settling in a position perpendicular to the tibial component; and
c. marking the final position of the P.T.T. over femoral condyle just anterior to the central watermark.

5. A method of using the orthopaedic surgical device—‘pegless template trial (PTT)’ of claim 1, for determining the mechanical axis, rotational position and size of the femoral component, comprising the steps of:
a) putting P.T.T. of appropriate size over the femoral condyle after tibial cut is taken and tibial insert is positioned;
b) moving the knee in extension to flexion multiple times & allowing settling of P.T.T, setting it right, making sure that this position stays clear of medial or lateral margins of medial femoral condyle & is 2 mm away all round, from cartilage-Patellofemoral at top & distal condylar on sides perpendicular to the tibial component;
c) optionally, inserting pins in the holes in P.T.T and removing the pins;
d) taking distal femoral cut and reinserting pins in the same hole; and
e) inserting femoral finishing block over the same two pins and posterior 105° cut, anterior cut and posterior chamfer cut are taken.

6. A method for determining ideal mechanical axis, rotational position, medio-lateral centering and size of the femoral component during partial knee replacement surgery comprising the steps of:
a) putting a template trial i.e. the “pegless template trial (PTT)” of appropriate size based on the surgical implant system being used over the femoral condyle after tibial cut is taken and tibial trial insert is positioned;
b) moving the knee through repetitive extension to flexion multiple times to see it settling in a position perpendicular to the tibial component; and
c) marking the final position of the P.T.T. over femoral condyle just anterior to the central watermark.

7. The method claim 6, wherein the ‘pegless template trial (PTT)’ comprises of: a thin metallic plate with a curved anterior end having two holes approximately of 2.5 mm diameter and a curved posterior end and a central watermark running longitudinally over the device and the said device having the same sagittal curve and mediolateral dimension as final femoral component of respective size ensuring that the femoral template sits over uncut femoral condyle and the holes of the said device matching the position of holes in femoral finishing block and the central watermark matching with the anterior notch of femoral finishing block enabling positioning of femoral component perpendicular to the tibial component and in centre of the medial femoral condyle.

8. The method claim 6, wherein the dimensions of the device are for Size 1 component are: total length—73 mm, thickness—1.5 mm, radius of the curved anterior end 9 mm, radius of the curvature of the curved posterior end 5.5 mm on both sides, distance between the holes 8 mm, distance of the holes from the anterior end, 4.5 mm.

9. An orthopaedic surgical device—‘pegless template trial (PTT)’ to determine mechanical axis, rotational position, medio-lateral centering and size of the femoral component substantially as shown and described in the drawings.
